(19) United States
(12) Patent Application Publication
(10) Pub. No.: US 2024/0245557 A1
(43) Pub. Date: Jul. 25, 2024

(54) DISPOSABLE PANT-TYPE GARMENT

(71) Applicant: Essity Hygiene and Health Aktiebolag, Göteborg (SE)

(72) Inventors: Lucas BÄCK, Göteborg (SE); Fanny STENHOLM, Göteborg (SE); Helena WALLIN, Göteborg (SE)

(73) Assignee: Essity Hygiene and Health Aktiebolag, Göteborg (SE)

(21) Appl. No.: 18/561,815

(22) PCT Filed: Jun. 9, 2021
(86) PCT No.: PCT/EP2021/065374

§ 371 Date: Nov. 17, 2023

(51) Int. Cl. A61F 13/494; A61F 13/49

(52) U.S. Cl. CPC A61F 13/49406; A61F 13/49012

(57) ABSTRACT

A disposable pant-type article including a front body panel and a rear body panel, each being elastically stretchable in the transverse direction, and a crotch region being located between a front panel crotch edge and a rear panel crotch edge. The article has first and second leg edges, each leg edge constituted by a front leg edge section being arranged in the front body panel, a rear leg edge section being arranged in the rear body panel; and a crotch leg edge section being arranged in the crotch region. A pre-tensioned first crotch elastic member is arranged extending in the longitudinal direction along the first leg edge and a pre-tensioned second crotch elastic member is arranged extending in the longitudinal direction along the second leg edge. The crotch leg edge section of the leg edges extends completely or partly in the respective crotch elastic member.

### TECHNICAL FIELD

The present disclosure pertains to a disposable pant-type article comprising front body panel and rear body panel the body panels being elastically stretchable in a transverse direction of the article, the front body panel having a front panel waist edge, a front panel crotch edge opposite the front panel waist edge and a first and a second front panel side edge and the rear body panel having a rear panel waist edge, a rear panel crotch edge opposite the rear panel waist edge and a first and a second rear panel side edge. The article has a crotch region comprising a crotch web material, the crotch region being arranged between the front body panel and the rear body panel and being contiguous with the front body panel along the front panel crotch edge and with the rear body panel along the rear panel crotch edge. The side edges of the front and rear body panels are joined in side seams so that the front panel waist edge and the rear panel waist edge together define a waist opening of the article and first and second leg edges of the article define first and second leg openings. The article further comprises a first and a second crotch elastic member, the crotch elastic members being arranged in the longitudinal direction along the first leg edge between the front body panel and the rear body panel.

### BACKGROUND

In the art of disposable pant-type articles and in particular disposable pant-type articles which are intended for use by adult users, the articles should meet high demands on fit, comfort and have a tailored and underwear like appearance while at the same time meeting equally high demands on absorbency and leakage protection. Disposable absorbent articles are articles which are intended to be discarded after a single use and not be laundered or otherwise restored for repeated use.

Pant-type articles of this kind are relatively complex products involving multiple components which are combined to form the absorbent article. Such components include nonwoven components, barrier components, elastic components and usually an absorbent core which may be provided in the form of an absorbent assembly including a barrier layer and a liquid permeable topsheet which together enclose the absorbent core. It is common to enhance body conformance and leakage security by arranging pre-tensioned elastic elements along all or selected parts of the waist opening and the leg openings of the pant-type article. The pre-tensioned elastic elements tend to gather the web materials to which they are attached and to form ruffled and wrinkled edge portions along the elastic elements. Such ruffled and wrinkled edge portions are undesirable as they may cause chafing especially in the groin area of a user. In addition, wrinkles which are created perpendicular to a leg edge may serve as channels through which body fluid may leak out of the article. Furthermore, the ruffled and wrinkled edge portions detract from an underwear-like appearance of the pant-type article and may be visible under ordinary clothes.

Accordingly, there exists a need for improved elastification of a disposable pant-type article.

### SUMMARY

Disposable pant-type articles having good functionality, fit and comfort, may be achieved at least in part by the features of claim 1. Variations of the disclosure are set out in the dependent claims.

Disclosed herein is a disposable pant-type article, the article having a longitudinal direction and a transverse direction, the article comprising:
a front body panel, the front body panel having a front panel waist edge, a front panel crotch edge opposite the front panel waist edge and a first and a second longitudinal front panel side edge;
a rear body panel, the rear body panel having a rear panel waist edge, a rear panel crotch edge opposite the rear panel waist edge and a first and a second longitudinal rear panel side edge;
a crotch region being located between the front panel crotch edge and the rear panel crotch edge, the crotch region being contiguous with the front body panel along the front panel crotch edge and being contiguous with the rear body panel along the rear panel crotch edge; wherein
the first front panel side edge is joined to the first rear panel side edge in a first side seam and the second front panel side edge is joined to the second rear panel side edge in a second side seam;
the front panel waist edge and the rear panel waist edge together define a waist opening of the article; and
a first leg edge defines a first leg opening and a second leg edge defines a second leg opening; wherein
the front body panel is elastically stretchable in the transverse direction, a front leg edge section of each of the first and the second leg edge being arranged in the front body panel web;
the rear body panel is elastically stretchable in the transverse direction, a rear leg edge section of each of the first and the second leg edge being arranged in the rear body panel web;
the crotch region comprises a crotch web material, a crotch leg edge section of each of the first and the second leg edge being arranged in the crotch region, a total length of the first leg edge being the combined length of the front leg edge section, the crotch leg edge section and the rear leg edge section of the first leg edge and a total length of the second leg edge being the combined length of the front leg edge section, the crotch leg edge section and the rear leg edge section of the second leg edge; and wherein
a pre-tensioned first crotch elastic member is arranged extending in the longitudinal direction along the first leg edge and a pre-tensioned second crotch elastic member is arranged extending in the longitudinal direction along the second leg edge, the crotch leg edge section of the first leg edge extending completely or partly in the first crotch elastic member and the crotch leg edge section of the second leg edge extending completely or partly in the second crotch elastic member.

The crotch region may comprise a crotch panel which is attached with a first side edge of the crotch panel to the front body panel along the front panel crotch edge, and with a second side edge of the crotch panel attached to the rear body panel along the rear panel crotch edge. The attachments between the crotch panel and the front and rear panels may be made as overlap seams extending along the front panel crotch edge and the rear panel crotch edge.

Alternatively, or in addition thereto, the crotch region may comprise a portion of a web material, such as an inner or outer cover web material of the pant-type article which extends continuously in the longitudinal direction from the front panel waist edge to the rear panel waist edge and bridges a gap between the front panel crotch edge and the rear panel crotch edge. The portion of the continuous cover web material which is applied in the crotch region constitutes the crotch web material.

In either case, the longitudinal extension of the crotch region is defined as the distance in the longitudinal direction between the front panel crotch edge of the elastically stretchable front body panel and the rear panel crotch edge of the elastically stretchable rear body panel. It may be preferred that the crotch web material extends over the entire surface of the crotch region.

In the disposable pant-type article as disclosed herein, the crotch elastic members each have a front end portion, wherein the front end portion of each of the first and the second crotch elastic members may overlap with and be attached to the front body panel and/or the rear end portion of each of the first and the second crotch elastic members may overlap with and be attached to the rear body panel. Thus, the first and second crotch elastic members may extend into the front body panel and/or into the rear body panel. Alternatively, the crotch elastic members are arranged only in the crotch region.

The crotch elastic members may be flat, band-shaped crotch elastic members.

The crotch elastic members may comprise or consist of elastic film or an elastic nonwoven.

The crotch elastic members may have a width in the transverse direction of the article in the range of from 15 mm to 50 mm, such as from 20 mm to 40 mm or from 25 mm to 35 mm, and a length in the longitudinal direction of the article in the range of from 150 mm to 350 mm, such as from 170 mm to 320 mm or from 200 mm to 310 mm. The dimensions of the crotch elastic members are measured with the pant-type article in a flat-out state with all elastic in the article in a full stretched out condition and with the side seams broken or cut open. In the fully stretched out condition the materials to which the crotch elastic members are attached are in a non-gathered state.

Each of the first crotch elastic member and the second crotch elastic member may have a narrowed intermediate portion, the narrowed intermediate portion of the crotch elastic member being formed by cutting away a portion of the material in the crotch elastic member, the narrowed intermediate portion of the crotch elastic member being arranged between the front end portion and the rear end portion of the crotch elastic member. Hence, the crotch elastic members may have a greater width in the front and rear end portions than in the intermediate portion. The narrow intermediate portion of the crotch elastic member has been formed by cutting away a portion of the crotch elastic member along the crotch leg edge, after the crotch elastic member has been applied to the pant-type article.

Each crotch elastic member thereby has a concave edge which at least partly coincides with the crotch leg edge. The term “concave edge”, as used herein should be broadly understood as including continuously curved concave edges and edges having an angular shape which changes in one or more distinctly defined steps as well as combinations of a continuous curvature and an angular shape. The concave edge may comprise one or more straight segments and optionally one or more curved segments. A concave crotch leg edge as referred to herein includes V-shaped and U-shaped crotch leg edges as well as any other shape providing the pant-type absorbent article with a crotch region which is narrower in a longitudinally intermediate part of the crotch region than at the end parts of the crotch region. A concave edge of a crotch elastic member which has been formed by simultaneous cutting through multiple layers of material in the crotch region, including one or more layers from a peripheral edge portion of an absorbent assembly, is coincident with a concave edge of the liquid permeable topsheet and/or with a concave edge of the liquid impermeable backsheet of the absorbent assembly.

The width of the front end portion may be equal to the width of the rear end portion. A ratio between the width of the end portions and the smallest width of the intermediate portion may be from 1:0.5 to 1:0.9.

Pre-tensioning of the crotch elastic members may suitably be achieved by attaching the crotch elastic members to the pant-type article with the crotch elastic members being in a stretched state. The crotch elastic member may be applied and attached to the pant-type article with the end portions of the crotch elastic members in a non-tensioned condition. The non-tensioned end portions of the crotch elastic members may each have a length of from 5% to 8% of the total length of the crotch elastic member, including the length of the end portions and the tensioned intermediate portion between the end portions.

A first reinforcing elastic feature may be arranged on the elastically stretchable rear body panel web along the rear leg edge section of the first leg edge and a second reinforcing elastic feature may be arranged on the elastically stretchable rear body panel web along the rear leg edge section of the second leg edge. The reinforcing elastic features are supplementary elastic elements which are applied in addition to the elastic material in the elastic body panels.

The first reinforcing elastic feature may extend along a first part of the rear leg edge section of the first leg edge in a direction away from a longitudinal centre line of the pant-type article towards the first side seam and may have an end point on the rear leg edge section of the first leg edge, with a second part of the rear leg edge section of the first leg edge being free from the first reinforcing elastic feature. The second part of the rear leg edge section of the first rear leg edge may have a length as measured along the first leg edge from the end point of the first reinforcing elastic feature to the first side seam which is from 25% to 75% of a total length of the rear leg edge section of the first leg edge.

The second reinforcing elastic feature may extend along a first part of the rear leg edge section of the second leg edge in a direction away from the longitudinal centre line of the pant-type article towards the second side seam and may have an end point on the rear leg edge section of the second leg edge, with a second part of the rear leg edge section of the second leg edge being free from the second reinforcing elastic feature. The second part of the rear leg edge section of the second leg edge may have a length as measured along the second leg edge from the end point of the second reinforcing elastic feature to the second side seam which is from 25% to 75% of a total length of the rear leg edge section of the second leg edge.

The first and second reinforcing elastic features may comprise multiple individual elastic elements, such as 2 to 15 individual elastic elements, 3 to 12 individual elastic element or 4 to 10 individual elastic elements.

The multiple individual elastic elements may be elastic strings, threads or bands which are applied spaced apart along the rear leg edge sections of the first and second leg edges.

The reinforcing elastic features may be covered with a nonwoven covering web.

The reinforcing elastic features may intersect with and be attached to the first and the second crotch elastic members. The first and second crotch elastic members may have been attached in the crotch region before or after attaching the reinforcing elastic features. As set out herein, the rear end portion of the first crotch elastic member and the rear end portion of the second crotch elastic member may overlap with and be attached to the elastic rear body panel. The reinforcing elastic feature may then be attached to the rear end portion of the first crotch elastic member and to the rear end portion of the second crotch elastic member. The rear end portions of the crotch elastic members may, thus, serve as anchoring areas for the reinforcing elastic features at the inner ends of the reinforcing elastic features.

It may be advantageous that the reinforcing elastic feature is attached to non-tensioned end portions of the crotch elastic members as a more secure attachment between the reinforcing elastic element and the crotch elastic members may be provided in this manner.

The crotch web material, which is applied in the crotch region, either as part of a coherent cover material or as a crotch panel material may be a non-elastic crotch web material, such as a non-elastic nonwoven web.

The pant-type article as disclosed herein may further comprise an absorbent core. The absorbent core may be part of an absorbent assembly and may be arranged between a liquid impermeable backsheet and a liquid permeable topsheet.

The absorbent assembly has side edges extending in a longitudinal direction of the pant-type article and end edges extending the transverse direction of the pant-type article. The absorbent assembly may further comprise reinforcing leg elastic elements applied along the side edges of the absorbent assembly.

The absorbent assembly may be applied to cover the stretched and attached crotch elastic members. In this manner the absorbent assembly will be overlaying the crotch elastic members on the inner surface of the pant-type article, the inner surface being the surface which is intended to be facing the wearer when the pant-type article is being worn. The inner surface of the absorbent assembly may be formed by the liquid permeable topsheet and may optionally include barrier cuffs extending along the side edges of the absorbent assembly.

It may be preferred that the topsheet of the absorbent assembly and any barrier cuffs are formed from fibrous web material, such as nonwoven material, providing the inner surface of the absorbent assembly with a textile character.

A part of each leg cut which extends in the crotch panel web may be cut through the crotch panel web, through the corresponding crotch elastic member and through a peripheral edge portion of the absorbent assembly, the peripheral edge portion of the absorbent assembly being formed by one or both of the liquid impermeable backsheet and the liquid permeable topsheet of the absorbent assembly, the peripheral edge portion of the absorbent assembly being arranged outward of a peripheral edge of the absorbent core of the absorbent assembly. In an absorbent assembly as disclosed herein, the topsheet and the backsheet may both extend beyond the periphery of the absorbent core and may be joined to each other in an edge seal extending along the periphery of the absorbent core outward of a peripheral edge of the absorbent core. A pant-type absorbent article which is produced in this way will have smooth non-wrinkled elastic leg edges which conform comfortably to the groin of a wearer. Hence, the formation of wrinkles which may act as channels through which fluid may leak out of the absorbent article is prevented or at least considerably reduced. Moreover, by using bandshaped crotch elastic members with generally uniform elastic properties over the width of the crotch elastic members, the forces from the crotch elastic members are distributed over the contact area between the crotch elastic members and a wearer's body which further contributes to improve wearer comfort.

The absorbent assembly may be applied to cover the crotch elastic members on an inner surface of the pant-type article, the inner surface being the surface which is intended to be facing the wearer when the pant-type article is being worn. In particular, the liquid impermeable backsheet and/or the liquid permeable topsheet may overlie the first and the second crotch elastic members. The inner surface of the absorbent assembly may be formed by the liquid permeable topsheet and may optionally include barrier cuffs extending along the side edges of the absorbent assembly.

It may be preferred that the topsheet of the absorbent assembly and any barrier cuffs are formed from fibrous web material, such as nonwoven material, providing the inner surface of the absorbent assembly with a textile character.

As set out herein, each crotch elastic member has a concavely curved edge which extends along the corresponding crotch leg edge section. The shaped edge of the crotch elastic member may be aligned with a shaped edge of the liquid permeable topsheet and/or with a shaped edge of the liquid impermeable backsheet, e.g., by having been cut simultaneously with the edge of the liquid permeable topsheet and/or with the edge of the liquid impermeable backsheet.

The liquid impermeable backsheet and/or the liquid permeable topsheet of the absorbent assembly may, thus, be coterminous with the first and the second crotch elastic members along at least a part of the crotch leg edge sections which extend into the crotch elastic members, such as along a part of each shaped crotch leg edge section which is arranged in a narrowed intermediate portion of the corresponding crotch elastic member.

The shaped part of each crotch leg edge section may be created by cutting through the crotch web material, through the crotch elastic member which extends along the crotch leg edge section and through a longitudinally extending peripheral edge portion of an absorbent assembly, if present, the peripheral edge portion of the absorbent assembly being formed by one or both of the liquid permeable topsheet and the liquid impermeable backsheet of the absorbent assembly, the peripheral edge portion of the absorbent assembly being arranged outward of a peripheral edge of the absorbent core of the absorbent assembly. In an absorbent assembly as disclosed herein, the topsheet and the backsheet may both extend beyond the periphery of the absorbent core and may be joined to each other in an edge seal extending along the periphery of the absorbent core outward of the peripheral edge of the absorbent core. The pant-type absorbent article having a crotch leg edge section in which the leg edge shape of the crotch elastic member has been created by the crotch elastic member being cut together with the crotch web material and preferably also together with a topsheet and/or a backsheet of an absorbent assembly provides the pant-type absorbent article with shaped, cuff-like, smooth elastic leg edges which conform comfortably to the groin of a wearer and which prevent formation of leakage-promoting wrinkles at the crotch leg edges.

In a disposable pant-type article as disclosed herein, the elastically stretchable front body panel web and/or the elastically stretchable rear body panel web may comprise or consist of an elastic laminate, the elastic laminate comprising first and second layers of fibrous material and an intermediate elastic layer arranged between the first and second layers of fibrous material.

The intermediate elastic layer may comprise or consists of a layer of elastic film.

It may be desirable that all sections of the first leg edge and the second leg edge are cut in an elastic web material, such that an elastic film, an elastic nonwoven or an elastic foam material, with a primary direction of the cut being in the direction of stretchability of the elastic material, i.e., the front and rear leg edge sections are cut primarily in the transverse direction and the crotch leg edge sections are cut primarily in the longitudinal direction.

In a disposable pant-type article as disclosed herein, the elastically stretchable front body panel web may form part of or constitute a continuous front body panel web which extends transversely across the width of the front portion between the first and the second front panel side edges and/or wherein the elastically stretchable rear body panel web forms part of or constitutes a continuous rear body panel web which extends transversely across the width of the rear portion between the first and the second rear panel side edges.

### BRIEF DESCRIPTION OF THE DRAWINGS

The pant-type articles as disclosed herein will be further explained hereinafter with reference to the appended drawings wherein:

FIG. 1 shows a planar view of a stretched-out absorbent pant-type article with open side seams, as seen from an inner surface of the pant-type article;

FIG. 2 shows an enlarged view of a central portion of the pant-type article in FIG. 1 with a first crotch leg edge shape;

FIG. 3 shows an enlarged view of a central portion of the pant-type article in FIG. 1 with a second crotch leg edge shape;

FIG. 4 shows the stretched-out pant-type article in FIG. 1 with the side seams closed; and

FIG. 5 shows the pant-type article in FIG. 4 in a non-stretched out condition, as it appears before use.

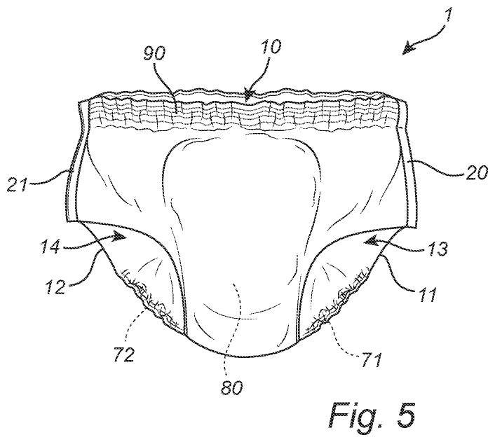

### DETAILED DESCRIPTION

It is to be understood that the drawings are schematic and that individual components or features, such as layers of material are not necessarily drawn to scale. The absorbent pant-type incontinence article for adult users which is shown in the figures is provided as an example only. By an “absorbent pant-type article” as used herein is implied a pant-type article that absorbs or is adapted to absorb bodily fluids, such as urine, blood and loose fecal matter.

Pant-type articles without an absorbent core or with an absorbent core which is not part of an absorbent assembly are also conceivable, as disclosed herein. Furthermore, the absorbent pant-type article may be a baby diaper, a training pant, a feminine hygiene pant, swimwear, disposable underpants, etc.

The pant-type article which is shown in the figures is a simplified article, and it should be understood that the article may contain further features, such as barrier cuffs. It is also to be understood that the waist elastic disclosed herein is optional or any other suitable type of waist elastic may be used. The side seams may be reclosable side seams, and the pant-type article may be provided with fastener elements to provide reclosability of the side seams.

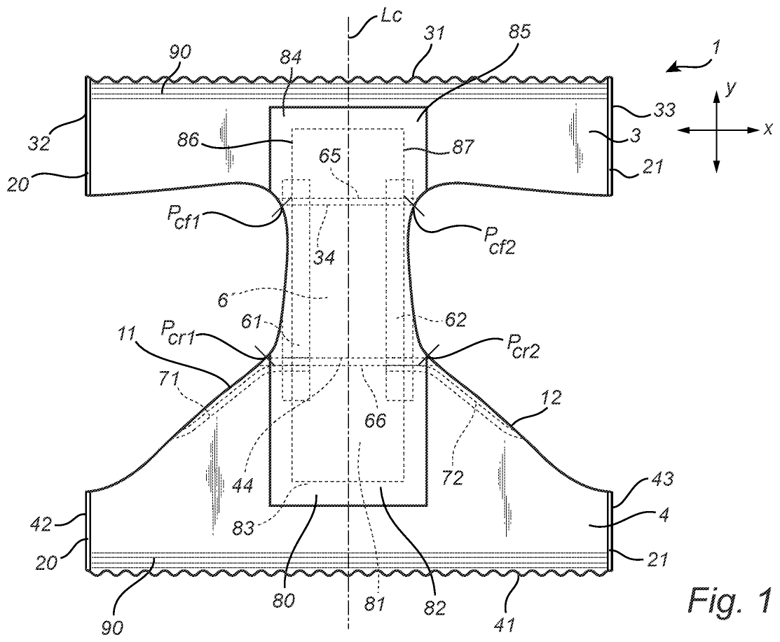

With initial reference to FIG. 1, there is shown a pant-type article 1 in the form of a pant-type incontinence article for adult users. The pant-type article 1 is shown in FIG. 1 in an unfolded and flat condition with all elastic elements in an extended state. The pant-type article has a longitudinal direction y and a transverse direction x.

The pant-type article 1 is seen from the inner surface which is the surface which will be facing a wearer's body when the article is being worn and which is opposite the outer, garment-facing surface of the pant-type article 1.

The pant-type article 1 comprises a front body panel 3, a rear body panel 4 and absorbent assembly 80 located in a crotch region 6 of the pant-type article 1 and extending in the longitudinal direction y forward in over the front body panel 3 and rearward in over the rear body panel 4. The absorbent assembly 80 bridges a gap between the front and rear body panels 3, 4. The absorbent assembly 80 is a separately produced component which comprises an absorbent core 81 which is enclosed between a liquid permeable topsheet 82 and a liquid impermeable backsheet 83.

The front body panel 3 has a front panel waist edge 31 extending in the transverse direction x, a pair of front panel side edges 32, 33 extending in the longitudinal direction y and a front panel crotch edge 34 extending in the transverse direction opposite the front panel waist edge 31. The rear body panel 4 has a rear panel waist edge 41 extending in the transverse direction x, a pair of rear panel side edges 42, 43 extending in the longitudinal direction y and a rear panel crotch edge 44 extending in the transverse direction x opposite the rear panel waist edge 41.

In the pant-type article 1 shown in the figures the elastically stretchable front body panel 3 has been formed from a continuous front body panel web which extends transversely across the width of the pant-type article 1 between the first and the second front panel side edges 32, 33 and the elastically stretchable rear body panel 4 has been formed from a continuous rear body panel web which extends transversely across the width of the pant-type article 1 between the first and the second rear panel side edges 42, 43.

The crotch region 6 is arranged between the front body panel 3 and the rear body panel 3 and is contiguous with the front body panel 3 along the front panel crotch edge 34 and is contiguous with the rear body panel 4 along the rear panel crotch edge 44. The extension of the crotch region 6 in the longitudinal direction y is defined as the distance in the longitudinal direction between the front panel crotch edge 34 and the rear panel crotch edge 44.

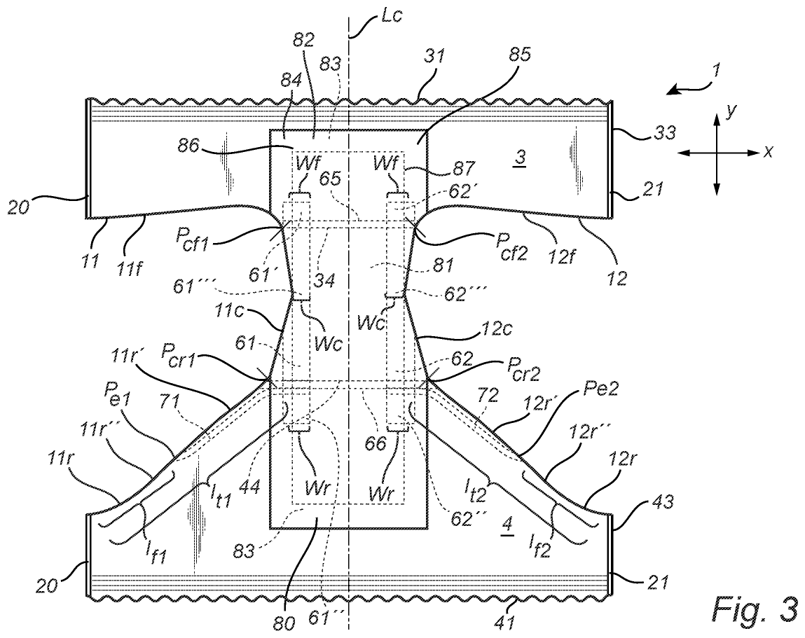

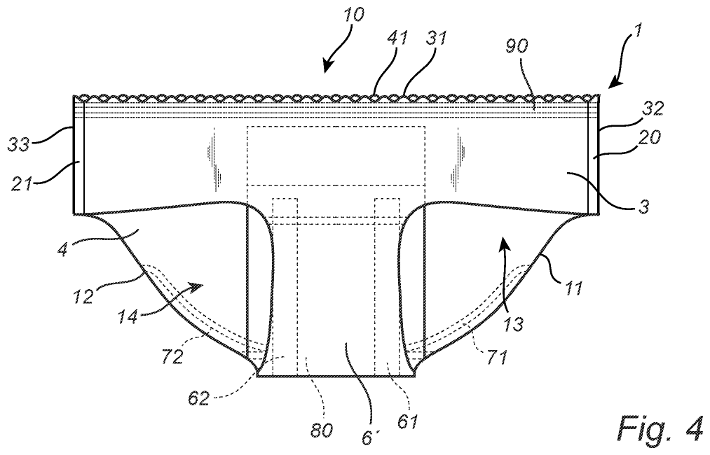

The first front panel side edge 32 is joined to the first rear panel side edge 42 in a first side seam 20 and the second front panel side edge 33 is joined to the second rear panel side edge 43 in a second side seam 21 to create the pant-type article 1 having a waist opening 10, a first leg opening 13 and a second leg opening 14, as shown in FIGS. 3 and 4.

The side seams of the pant-type articles as disclosed herein may be generally band-shaped joins which are formed by ultrasonic welding or thermo welding. In order to have sufficient strength to withstand the forces to which the pant-type article is exposed during donning of the article and to allow sufficient production tolerances when cutting off individual pant-type garments from a precursor web, the side seams commonly have a width in the order of 5 to 10 millimetres. It is also known to make side seams having a width less than 5 millimetres.

It is generally desired that a soiled pant-type garment can be easily removed without having to pull the garment down over the legs of a user. Therefore, the side seams are commonly made such that they are breakable by manual force to allow a user or a caregiver to pull apart the side seams before removing a soiled pant-type garment.

The waist opening 10 is defined by the front panel waist edge 31 and the rear panel waist edge 41. A first leg edge 11 defines the first leg opening 13 and a second leg edge 12 defines the second leg opening 14.

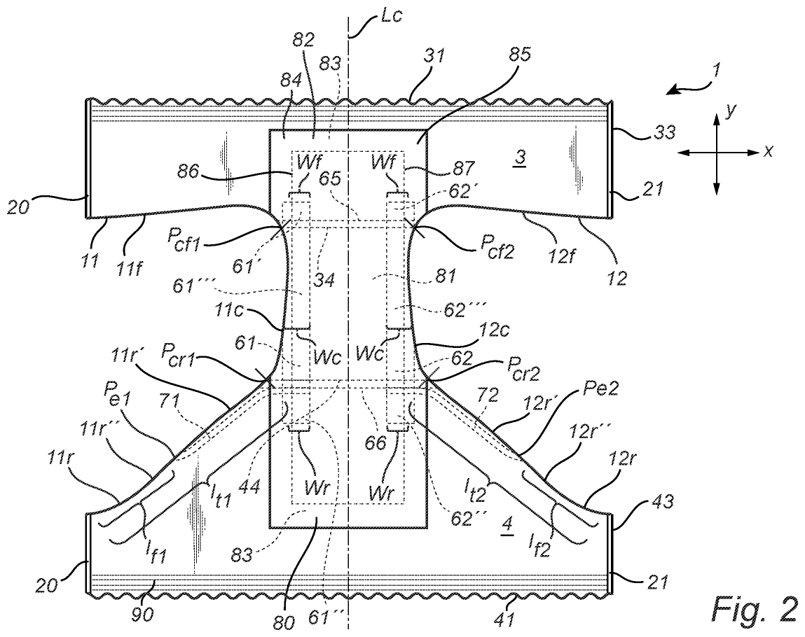

The front body panel 3 comprises a front body panel web which is elastically stretchable at least in the transverse direction x and which provides the overall elastic stretchability of the front body panel 3. A front leg edge section 11f, 12f of each of the first and the second leg edge 11, 12, is arranged in the elastically stretchable front body panel web 3 as shown in FIG. 2.

The rear body panel 4 comprises a rear body panel web which is elastically stretchable at least in the transverse direction x and which provides the overall elastic stretchability of the rear body panel 4. A rear leg edge section 11r, 12r of each of the first and the second leg edge 11, 12, is arranged in the elastically stretchable rear body panel web 4 as shown in FIG. 2.

The crotch region 6 comprises a crotch web material, preferably a non-elastic crotch web material, such as a non-elastic nonwoven web. A crotch leg edge section 11c, 12c of each of the first and the second leg edge 11, 12 is arranged in the crotch web material.

The total length of the first leg edge is the combined length of the front leg edge section 11f, the crotch leg edge section 11c and the rear leg edge section 11r of the first leg edge 11 and a total length of the second leg edge 12 is the combined length of the front leg edge section 12f, the crotch leg edge section 12c and the rear leg edge section 12r of the second leg edge 12.

A first crotch elastic member 61 is arranged extending in the longitudinal direction y along the first leg edge 11 between the front panel crotch edge 34 and the rear panel crotch edge 44 and a second crotch elastic member 62 is arranged extending in the longitudinal direction y along the second leg edge 12 between the front panel crotch edge 34 and the rear panel crotch edge 44. The crotch elastic members 61, 62 are constituted by band-shaped strips of elastic film which are applied to the pant-type article in a stretched condition and form functional leg elastic in the crotch region of the pant-type article 1.

The crotch leg edge sections 11c, 12c are cut into the crotch elastic members 61, 62, whereby the front end portions 61′, 62′ of the crotch elastic members 61, 62 and the rear end portions 61″, 62″ of the crotch elastic members 61, 62 have a greater width wf, wr in the front and rear end portions 61′, 62′, 61″, 62″ of the crotch elastic members 61, 62 than a smallest width wc of the intermediate portion 61′″, 62′″ of the crotch elastic members.

In FIGS. 1 and 2, the crotch leg edge sections 11c, 12c are shown with a slightly concave curvature, such that also the band-shaped crotch elastic members 61, 62 have a somewhat modified band-shape with a concave cut edge along at a part of the crotch leg edge sections 11c, 12c. A further example of a concave shape of the crotch leg edge sections 11c, 12c is shown in FIG. 3. The crotch leg edge sections 11c, 12c have the shape of a shallow V with the bottom of the V placed at the narrowest part of the crotch region 6, which is also where the crotch elastic members 61, 62 have their smallest width, WC.

In the pant-type article 1 which is shown in the Figures, the width wf of the front end portions 61′, 62′ of the crotch elastic members 61, 62 is equal to the width wr of the rear end portion 61″, 62″. The front end portions 61′,62′ and the rear end portions 61″, 62″ have a greater width than the narrowest width of the intermediate portion 61″, 62′″ of the crotch elastic members 61,62. A suitable ratio between the width of the end portions wf, wr of the crotch elastic members 61,62 and the smallest width wc of the intermediate portion of the crotch elastic members 61,62 may be from 1:0.5 to 1:0.9.

It may be desirable that all sections 11f, 12f, 11c, 12c, 11r, 12r of the first leg edge 11 and the second leg edge 12 are cut in a coherent elastic material, such that an elastic film, an elastic nonwoven or an elastic foam material, with a primary direction of the cut being in the direction of stretchability of the elastic material, i.e., the front and rear leg edge sections 11f, 12f, 11r, 12r are cut primarily in the transverse direction x and the crotch leg edge sections 11c, 12c are cut primarily in the longitudinal direction y.

The crotch elastic members 61, 62 each have a front end portion 61′, 62′ at the front panel crotch edge 34, a rear end portion 61″, 62″ at the rear panel crotch edge 44 and an intermediate portion 61″, 62′″ between the front end portion 61′, 62′ and the rear end portion 61″, 62″. The front end portion 61′, 62′ of each of the first and the second crotch elastic members 61, 62 overlaps with and is attached to the front body panel 3 and the rear end portion 61″, 62″ of each of the first and the second crotch elastic members 61, 62 overlaps with and is attached to the rear body panel 4. The end portions 61′, 62′, 61″, 62″ of the crotch elastic members 61, 62 may be non-tensioned parts of the crotch elastic members 61, 61. Non-tensioned end portions 61′, 62′, 61″, 62″ of the crotch elastic members 61, 62 may each have a length of from 5% to 8% of the total length of the crotch elastic member, including the length of the end portions 61′, 62′, 61″, 62″ and the pre-tensioned intermediate portion 61″, 62″ located between the end portions 61′, 62′, 61″, 62″.

The crotch region 6 is formed by a crotch panel 6′ which is attached with a first side edge 65 of the crotch panel 6′ to the front body panel 3 along the front panel crotch edge 34, and with a second side edge 66 of the crotch panel 6′ attached to the rear body panel 4 along the rear panel crotch edge 44. The attachments between the crotch panel and the front and rear panels are made as overlap seams.

Alternatively, or in addition to applying a crotch panel in the crotch region as shown in the Figures, the crotch region may comprise a portion of a web material, such as an inner or outer cover web material of the pant-type article which extends continuously in the longitudinal direction from the front panel waist edge to the rear panel waist edge and bridges a gap between the front panel crotch edge and the rear panel crotch edge. The portion of the continuous cover web material which is applied in the crotch region constitutes the crotch web material.

A first reinforcing elastic feature 71 is arranged on the elastically stretchable rear body panel 4 along the rear leg edge section 11r of the first leg edge 11 and a second reinforcing elastic feature 72 is arranged on the elastically stretchable rear body panel 4 along the rear leg edge section 12r of the second leg edge 12. The first reinforcing elastic feature 71 extends along a first part 11r′ of the rear leg edge section 11r of the first leg edge 11 in a direction away from a longitudinal centre line Lc of the pant-type article 1 towards the first side seam 20 and having an end point Pe1 on the rear leg edge section 11r of the first leg edge 11, with a second part 11r″ of the rear leg edge section 11r of the first leg edge 11 being free from the first reinforcing elastic feature 71.

The second part 11r″ of the rear leg edge section 11r of the first rear leg edge 11 which is free from reinforcing elastic, has a length If1 as measured along the first leg edge 11 from the end point Pe1 of the first reinforcing elastic feature 71 to the first side seam 20 which is from 25% to 75% of a total length It1 of the rear leg edge section 11r of the first leg edge 1.

The second reinforcing elastic feature 72 extends along a first part 12r′ of the rear leg edge section 12r of the second leg edge 12 in a direction away from the longitudinal centre line Lc of the pant-type article 1 towards the second side seam 21 and has an end point Pe2 on the rear leg edge section 12r of the second leg edge 12, with a second part 12r″ of the rear leg edge section 12r of the second leg edge 12 being free from the second reinforcing elastic feature 72, the second part 12r″ of the rear leg edge section 12r of the second leg edge 12 having a length If2 as measured along the second leg edge 12 from the end point Pe2 of the second reinforcing elastic feature 72 to the second side seam 21 which is from 25% to 75% of a total length It2 of the rear leg edge section 12r of the second leg edge 12.

With reference to FIGS. 1 and 2, the pant-type article 1 further comprises an absorbent assembly 80 comprising an absorbent core 81, a liquid permeable topsheet 82 and a liquid impermeable backsheet 83, the absorbent core 81 being enclosed between the liquid permeable topsheet 82 and the liquid impermeable backsheet 83.

The absorbent core 81 shown in the figures has a rectangular design. However, the disclosure is not limited to rectangular cores and it is to be understood that the absorbent core may have any useful shape, such as hourglass shape T-shape, etc., as known in the art.

The absorbent assembly 80 is applied on the inner surface of the pant-type article 1, the inner surface being the surface which is intended to be facing the wearer when the pant-type article 1 is being worn. The absorbent assembly 80 covers the pre-tensioned crotch elastic members 61, 62. The inner surface of the absorbent assembly is formed by the liquid permeable topsheet 82 and may optionally include barrier cuffs (not shown) extending along the side edges of the absorbent assembly 80.

A part of each leg edge 11, 12 which extends in the crotch region 6 has been formed by a cut through the material in the crotch panel 6′, through the corresponding crotch elastic member 61, 62 and through a peripheral edge portion 84, 85 of the absorbent assembly 80.

The peripheral edge portions 84, 85 of the absorbent assembly 80 are formed by edge parts of the liquid permeable topsheet 82 and the liquid impermeable backsheet 83 which extend in the transverse direction x outward of the peripheral side edges 86, 87 of the absorbent core 81 of the absorbent assembly 80.

As disclosed herein, the topsheet 82 and the backsheet 83 may both extend beyond the periphery of the absorbent core 81 and may be joined to each other in an edge seal extending along the periphery of the absorbent core outward of a peripheral edge of the absorbent core. In the embodiment shown in the Figs., the topsheet 82 and the backsheet 83 are only joined in edge seals in the peripheral edge portions 84, 85 which extend along the peripheral side edges 86, 87 of the absorbent core 81. However, such edge seals may be arranged along the complete periphery of the absorbent core 81, i.e., also along the transverse end edges of the absorbent core 81. Furthermore, the peripheral edge portion 84, 85 of the absorbent assembly 80 may be formed by only one of the topsheet and the backsheet, as set out herein.

The absorbent assembly 80 covers the crotch elastic members 61, 62 on the inner surface of the pant-type article 1 with the peripheral edge portions 84, 85 of the absorbent assembly 80 which are formed by the topsheet 82 and the backsheet 83 overlying a portion of the respective crotch elastic member 61, 62 adjacent the corresponding crotch leg edge section 11c, 12c.

As set out herein, the crotch leg edge sections 11c, 12c have a concave shape as defined herein, the shape of the crotch leg edge sections 11c, 12c having been formed by cutting through the superposed crotch web material, the crotch elastic members 61, 62 and the topsheet 82 and the backsheet 83 in the peripheral edge portion 84, 85 along each crotch leg edge section 11c, 12c. As shown in FIGS. 1, 2 and 3, the cuts have been made such that a small part of each crotch elastic member 61, 62 has been cut away in the intermediate portion 61″, 62′″ of the crotch elastic member 61, 62, providing the intermediate portion 61″, 62′″ of the crotch elastic member 61, 62 with a shaped edge which is coterminous with the simultaneously formed cut edges of the crotch web material, the topsheet 82 and the backsheet 83. In the embodiment shown in the figures, the crotch web material is the web material which forms the crotch panel 6′. However, in other embodiments, the crotch web material may be formed by a portion of an outer cover web, as set out herein.

An absorbent core 81 as disclosed herein may comprise any conventional material suitable for absorbing discharged bodily wastes, such as cellulosic fluff pulp, tissue layers, highly absorbent polymers (super absorbents), absorbent foam materials including hydrogel-foam material, absorbent nonwoven materials, or the like. The absorbent core can comprise non-absorbent components such as stiffening elements, shaping elements, binders, etc. Various types of liquid-receiving and liquid distribution elements can also be included in the core.

The liquid permeable topsheet 82 may comprise or consist of a nonwoven material. Other suitable topsheet materials include tow fibres, porous foams, apertured plastic films and laminates and combinations of such materials. The materials which are best suited as topsheet materials are soft and non-irritating to the skin, are readily penetrated by body fluids, and display low rewet.

The liquid impermeable backsheet 83 may consist of a thin plastic film, e. g. a polyethylene or polypropylene film, a nonwoven material coated with a liquid impervious material, a hydrophobic nonwoven material which resists liquid penetration or laminates of plastic film and nonwoven. The backsheet material may be breathable to allow vapour to escape from the absorbent body, while still preventing liquids from passing through the backsheet material.

The topsheet and backsheet may be connected to each other for example by adhesive bonding, gluing or welding by heat or ultrasonically. The topsheet and/or the backsheet may further be attached to the absorbent core by any method known in the art, such as adhesive, heat-bonding, welding, needling, etc.

The pant-type articles as disclosed herein may be of the kind shown in the Figures and having a two-part chassis with a crotch panel which is connected to the front panel along the front panel crotch edge and which is connected to the rear panel along the rear panel crotch edge. Alternatively, the pant-type article may have a unitary chassis having a non-elastic outer or inner cover web extending the full distance between the front panel waist edge and the rear panel waist edge, the cover web constituting a non-elastic layer of each of the front panel and the rear panel and constituting the crotch web material in the crotch region of the pant-type article.

The elastically stretchable front body panel and the elastically stretchable rear body panel may comprise or consist of a stretchbonded laminated elastic web material.

Suitable stretch-bonded laminates, may comprise nonwoven material layers or webs such as spunbond, air laid, wet laid, carded, electro spun or meltblown nonwovens. The nonwoven material may be bonded by any suitable technique, such as by needling, hydroentangling, ultrasonic welding, or thermobonding.

The fibers of the nonwoven materials used herein may be man-made fibers, natural fibers or mixtures of man-made and natural fibers. Man-made fibers include mono-component, bi-component and multicomponent fibers of polymers such as polyolefins, polyesters, polyacrylates, etc., as well as regenerated fibers such as viscose fibers and modal fibers. Natural fibres are for instance cellulosic fibres such as pulp fibers, cotton fibers, flax, hemp, etc.

The pant-type article 1 as disclosed herein may have an elastic waist feature 90 arranged along the waist opening 10. An elastic waist feature 90 may be formed by one or more elastic elements extending parallel with the front panel waist edge 31 and the rear panel waist edge 41. The elastic waist element or elements may be incorporated in the front body panel 3 and the rear body panel 4 or may be applied as a separate waistband which is attached to the front panel waist edge 31 and the rear panel waist edge 41. The pant-type article 1 which is shown in the Figures has an elastic waist feature 90 which extends around the full circumference of the waist opening 10. However, an elastic waist feature may be arranged only along a part of the waist opening, such as only along the rear panel waist edge, only along the front panel waist edge or along a part of one or both the front and the rear panel waist edge which part has a length which is less than the full length of the corresponding panel waist edge.

The elastic material in elastic elements arranged along the leg openings 13, 14 and the waist opening 10 as disclosed herein may be any suitable elastic material such as natural or synthetic rubber, thermoplastic elastomers, such as thermoplastic polyurethane or styrene block co-polymers or elastane, also referred as to spandex (polyurethane-polyurea copolymer). The elastic elements may be of the elastane type that is available under the trade name “LYCRA”, but any suitable elastic thread may be used. The threads may have a linear mass density, dtex, of about 80-1200 dtex.

The nonwoven web-materials used in the pant-type articles as disclosed herein may comprise thermoplastic material. The nonwoven web-materials will typically be incorporated in joins and seams in the pant-type article and it is desirable that the nonwoven webs be weldable by heat or by ultrasonic welding processes. Examples of suitable polymers for use in the fibrous nonwoven webs as disclosed herein are polyethylene polypropylene and other polyolefin homopolymers and copolymers and polyesters. The weldable nonwoven webs have a high content of thermoplastic component and preferably contain at least 50% thermoplastic fibres and more preferably at least 80% thermoplastic fibres.

The elastic material used in the front and rear body panels is preferably a breathable elastic film such as an apertured or microporous elastic film. The elastic film may have a basis weight of between 20 and 80 g/m², preferably between 20 and 60 g/m². The elastic web may be of any suitable elastic polymer, natural or synthetic. Some examples of useful materials for the elastic web are low crystallinity polyethylenes, metallocene-catalyzed low crystallinity polyethylenes, ethylene vinyl acetate copolymers (EVA), polyurethane, polyisoprene, butadiene-styrene copolymers, styrene block copolymers, such as styrene/isoprene/styrene (SIS), styrene/butadiene/styrene (SBS), or styrene/ethylene-butadiene/styrene block copolymer. Blends of these polymers may also be used as well as other modifying elastomeric or non-elastomeric materials. The total basis weight of the stretchbonded elastic laminate web in a relaxed condition is preferably between 40 and 100 g/m², more preferably not more than 90 g/m².

1. A disposable pant-type article, the article having a longitudinal direction and a transverse direction, the article comprising:
a front body panel, the front body panel having a front panel waist edge, a front panel crotch edge opposite the front panel waist edge and a first and a second longitudinal front panel side edge;
a rear body panel, the rear body panel having a rear panel waist edge, a rear panel crotch edge opposite the rear panel waist edge and a first and a second rear panel side edge;
a crotch region being located between the front panel crotch edge and the rear panel crotch edge, the crotch region being contiguous with the front body panel along the front panel crotch edge and being contiguous with the rear body panel along the rear panel crotch edge; wherein
the first front panel side edge is joined to the first rear panel side edge in a first side seam and the second front panel side edge being joined to the second rear panel side edge in a second side seam;
the front panel waist edge and the rear panel waist edge together define a waist opening; and
a first leg edge defines a first leg opening and a second leg edge defines a second leg opening, wherein
the front body panel is elastically stretchable in the transverse direction, a front leg edge section of each of the first and the second leg edge being arranged in the front body panel;
the rear body panel is elastically stretchable in the transverse direction, a rear leg edge section of each of the first and the second leg edge being arranged in the rear body panel;
the crotch region comprises a crotch web material, a crotch leg edge section of each of the first and the second leg edge being arranged in the crotch region;
wherein
a total length of the first leg edge is the combined length of the front leg edge section, the crotch leg edge section and the rear leg edge section of the first leg edge and a total length of the second leg edge is the combined length of the front leg edge section, the crotch leg edge section and the rear leg edge section of the second leg edge; and
wherein
a pre-tensioned first crotch elastic member is arranged extending in the longitudinal direction along the first leg edge and a pre-tensioned second crotch elastic member is arranged extending in the longitudinal direction along the second leg edge, the crotch leg edge section of the first leg edge extending completely or partly in the first crotch elastic member and the crotch leg edge section of the second leg edge extending completely or partly in the second crotch elastic member.

2. A disposable article according to claim 1, wherein the crotch elastic members each have a front end portion and a rear end portion and wherein the front end portion of each of the first and the second crotch elastic members overlaps with and is attached to the front body panel and/or the rear end portion of each of the first and the second crotch elastic members overlaps with and is attached to the rear body panel.

3. A disposable article according to claim 1, wherein each of the first crotch elastic member and the second crotch elastic member has a narrowed intermediate portion, the narrowed intermediate portion of the crotch elastic member being formed by cutting away a portion of the material in the crotch elastic member, the narrowed intermediate portion of the crotch elastic member being arranged between the front end portion and the rear end portion of the crotch elastic member.

4. A disposable article according to claim 3, wherein the width of the front end portion of the crotch elastic member is equal to the width of the rear end portion of the crotch elastic member and a ratio between the width of the end portions and the smallest width of the intermediate portion of the crotch elastic member is from 1:0.5 to 1:0.9.

5. A disposable article according to claim 1, wherein the crotch elastic members comprise or consist of elastic film, or elastic nonwoven.

6. A disposable article according to claim 1, wherein a first reinforcing elastic feature is arranged on the elastically stretchable rear body panel along the rear leg edge section of the first leg edge and a second reinforcing elastic feature is arranged on the elastically stretchable rear body panel along the rear leg edge section of the second leg edge, the first reinforcing elastic feature extending along a first part of the rear leg edge section of the first leg edge in a direction away from a longitudinal centre line of the pant-type article towards the first side seam and having an end point on the rear leg edge section of the first leg edge, with a second part of the rear leg edge section of the first leg edge being free from the first reinforcing elastic feature, the second part of the rear leg edge section of the first leg edge having a length as measured along the first leg edge from the end point of the first reinforcing elastic feature to the first side seam which is from 25% to 75% of a total length of the rear leg edge section of the first leg edge, the second reinforcing elastic feature extending along a first part of the rear leg edge section of the second leg edge in a direction away from the longitudinal centre line of the pant-type article towards the second side seam and having an end point on the rear leg edge section of the second leg edge, with a second part of the rear leg edge section of the second leg edge being free from the second reinforcing elastic feature, the second part of the rear leg edge section of the second leg edge having a length as measured along the second leg edge from the end point of the second reinforcing elastic feature to the second side seam which is from 25% to 75% of a total length of the rear leg edge section of the second leg edge.

7. A disposable article according to claim 6, wherein the reinforcing elastic features intersect with and are attached to the first and the second crotch elastic members.

8. A disposable article according to claim 1, wherein the crotch web material is a non-elastic crotch web material, such as a non-elastic nonwoven web.

9. A disposable article according to claim 1, wherein the article further comprises an absorbent assembly comprising an absorbent core, a liquid permeable topsheet and a liquid impermeable backsheet, the absorbent core being enclosed between the liquid permeable topsheet and the liquid impermeable backsheet.

10. A disposable article according to claim 9, wherein the liquid permeable topsheet and/or the liquid impermeable backsheet overlies the first and the second crotch elastic members.

11. A disposable article according to claim 10, wherein each crotch elastic member has a shaped edge extending along the corresponding crotch leg edge section, the shaped edge of the crotch elastic member being aligned with a shaped edge of the liquid permeable topsheet and/or with a shaped edge of the liquid impermeable backsheet-.

12. A disposable pant-type article according to claim 1, wherein the elastically stretchable front body panel and/or the elastically stretchable rear body panel comprises or consists of an elastic laminate, the elastic laminate comprising at least a first layer of fibrous material and an elastic layer.

13. A disposable pant-type article according to claim 12, wherein the elastic laminate comprises a second layer of fibrous material and the elastic layer is an intermediate elastic layer arranged between the first and second layers of fibrous material.

14. A disposable pant-type article according to claim 12, wherein the elastic layer comprises or consists of a layer of elastic film.

15. A disposable pant-type article according to claim 12, wherein the elastic layer comprises or consists of a layer of elastic nonwoven material.

16. A disposable article according to claim 1, wherein the elastically stretchable front body panel is formed from a continuous front body panel web which extends transversely across the width of the pant-type article between the first and the second front panel side edges and/or wherein the elastically stretchable rear body panel is formed from a continuous rear body panel web which extends transversely across the width of the pant-type article between the first and the second rear panel side edges.
